SEQUENCE LISTING (7 sequences)

SEQ ID NO: 1 - DNA, 477 bp, Homo sapiens
  source 1..477: mol_type = genomic DNA
atgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaa

SEQ ID NO: 2 - protein, 158 aa, Homo sapiens
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 3 - DNA, 297 bp, Homo sapiens
  source 1..297: mol_type = genomic DNA
atgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataa

SEQ ID NO: 4 - protein, 98 aa, Homo sapiens
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 5 - DNA, 23 bp, synthetic construct
  source 1..23: mol_type = other DNA
ggagagcttggctctgttggggg

SEQ ID NO: 6 - DNA, 816 bp, synthetic construct
  source 1..816: mol_type = other DNA
ccggccatttcactctgactacatcatgtcaccaaacatctgatggtcttgccttttaattctcttttcgaggactgagagggagggtagcatggtagttaagagtgcaggcttcccgcattcaaaatcggttgcttactagctgtgtggctttgagcaagttactcaccctctctgtgcttcaaggtccttgtctgcaaaatgtgaaaaatatttcctgcctcataaggttgccctaaggattaaatgaatgaatgggtatgatgcttagaacagtgattggcatccagtatgtgccctcgaggcctcttaattattactggcttgctcatagtgcatgttctttgtgggctaactctagcgtcaataaaaatgttaagactgagttgcagccgggcatggtggctcatgcctgtaatcccagcattctaggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattccccca

SEQ ID NO: 7 - DNA, 877 bp, synthetic construct
  source 1..877: mol_type = other DNA
acagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaa

(19) United States
(12) Patent Application Publication
(10) Pub. No.: US 2025/0270509 A1
(43) Pub. Date: Aug. 28, 2025

(54) IMMORTALIZED CELLS FOR PRODUCTION OF BIOPRODUCTS, METHODS OF PRODUCING BIOPRODUCTS WITH AN IMMORTALIZED CELL, AND METHODS OF MAKING IMMORTALIZED CELLS FOR PRODUCTION OF BIOPRODUCTS

(71) Applicant: John Paul II Medical Research Institute, Coralville, IA (US)

(72) Inventors: Anant KAMATH, Coralville, IA (US); Alan B. MOY, Coralville, IA (US); Sara TERNES, Coralville, IA (US)

(73) Assignee: John Paul II Medical Research Institute, Coralville, IA (US)

(21) Appl. No.: 18/857,595

(22) PCT Filed: Apr. 18, 2023
(86) PCT No.: PCT/US2023/065911

§ 371 Date: Oct. 17, 2024

Related U.S. Application Data
(60) Provisional application No. 63/332,092, filed on Apr. 18, 2022.

(51) Int. Cl. C12N 5/0775; C07K 14/005; C07K 14/715; C12N 5/0789

(52) U.S. Cl. CPC C12N 5/0662; C07K 14/005; C07K 14/7158; C12N 5/0647; C12N 2510/04; C12N 2710/20022; C12N 2750/14122

(57) ABSTRACT

Described herein is an immortalized mammalian cell configured for production of a bioproduct of interest, which cell has a safe harbor locus selected from an Adeno Associated Virus 1 (AAVS1) locus, a CCR5 locus, and a Rosa26 locus into which a Human Papillomavirus (HPV) E6 gene sequence and an HPV E7 gene sequence have been inserted. Also disclosed herein are methods for immortalizing a mammalian cell for production of a bioproduct of interest as well as methods of producing a bioproduct of interest.

### CLAIM OF PRIORITY

The present application claims priority to U.S. Provisional Application No. 63/332,092, filed Apr. 18, 2022, which application is incorporated by reference herein in its entirety.

### FIELD OF INVENTION

The invention relates to the field of molecular and cell biology and the production of cell lines and biologics for various uses including in therapy and medical applications.

### BACKGROUND

Viruses have long been used to immortalize cells (See, e.g., Henle et al., Annals of the New York Academy of Sciences 354:326-31, 1980). As an example, the Adenovirus E1 gene has been used to immortalize Human Embryonic Kidney (HEK) 293 cells. However, infection of the target cell with the immortalizing virus often results in transfer of large stretches of DNA from the virus to the target cell (See, e.g., Xu et al., Cytotechnology 51 (3): 133-140, 2006).

As a result, there is a risk of release of replication competent virus from the immortalized cell when such a cell is further transfected with vectors and/or other nucleic acid constructs for production of bioproducts.

Prior methods of large-scale production of biologics have relied on various cell types, including bacteria and yeast cells, as well as mammalian cells such as CHO cells, PERC.6 cells, HEK 293 cells (See, e.g., Genzel, Biotechnology Journal 10 (5): 728-740, 2015; and U.S. Pat. No. 8,889,941). However, the type of cell influences the glycosylation state and/or other post-translational modifications of the biologic, which in turn regulate the pharmacologic properties of the bioproducts produced in the cell, particularly proteins. For example, glycosylation is known to affect protein folding, stability, solubility, protein-protein interactions, in vivo bioavailability, bio-distribution, pharmacokinetics, immunogenicity, and protein activity (See, e.g., Kaushik et al., Protein Science 20 (3): 465-81, 2011; Kayser et al., Biotechnology Journal 6 (1): 38-44, 2011; Li et al., Current Opinion in Biotechnology 20 (6): 678-84, 2009; Oberg et al., The Journal of Biological Chemistry 286 (36): 31915-23, 2011; Opanasopit et al., Am J Physiol Gastrointest Liver Physiol 280 (5): G879-89, 2001; Rajagopalan et al., Journal of the Association for Research in Otolaryngology 11(1): 39-51, 2010; Straumann et al., The Biochemical Journal 393(Pt 2): 459-69, 2006; Su et al., International Journal of Hematology 91(2): 238-44, 2010). Also, there are differences in glycosylation patterns in proteins between CHO, HEK 293 cells, and human plasma. For example, the glycosylation pattern for factor VII when produced from HEK 293 cells differs significantly from plasma-derived factor VII (See, e.g., Croset et al. Journal of Biotechnology 161(3): 336-48, 2012; Bohm et al. BMC Biotechnology 15:87, 2015).

HEK 293 cells pose additional significant shortcomings. Notwithstanding that the cell line is ethically controversial and is causing drug hesitancy among many individuals in view of its derivation, HEK 293 cells have a demonstrated aneuploidy karyotype, exhibiting 64 chromosomes (See, e.g., Lin et al., Nature Communications 5:4767, 2014). Moreover, HEK 293 cells were generated by transfection of cultured human embryonic kidney cells with sheared adenovirus-5 DNA and subsequent analysis has shown that the transformation resulted in the insertion of 4.5 kilobases from the left arm of the viral genome into chromosome 19 (See, e.g., Graham et al., The Journal of General Virology 36 (1): 59-74, 1977; Louis et al, Virology 233 (2): 423-9, 1997). HEK 293 cells therefore also pose a small risk of introducing a replication competent virus when further transfected with viral vectors. Use of HEK 293 cells also comes with the risk of introduction of trace amounts of contaminating viral proteins when producing a biologic.

Additionally, while immortalized human cell lines like the HEK 293 and PER. C6 may be used to produce replication-deficient viral vectors for gene therapy, the plasma membrane of these established human cell lines express an indeterminant array of surface proteins/receptors of viral origin that may have undesirable safety issues. Moreover, CHO cells, HEK 293 and PER. C6 cells possess a significant shortcoming insofar as these cell lines cannot be replicated again from their parental cell lines. Thus, the ability to maintain consistent and reliable cell-based bio-production—essential to the manufacture of medical agents where efficacy and safety are paramount—is compromised with these cell lines.

In view of the above, there is a need for cell lines configured for production of bioproducts, which cell lines avoid the safety hazards posed by possible release of replication competent viruses and which can consistently and reliably produce proteins and other biologics of interest free of viral contaminants and having native human post-translational modification states.

Postnatal somatic stem cells are an attractive and reliable source to produce immortalized human cell lines for at least a few reasons. First, postnatal somatic stem cells are free of adventitious agents. Second, the cell phenotype is typically uniform because the donor has not been subjected to years of environmental exposures or have been subjected to chronic disease. However, the doubling time of postnatal somatic stem cells is typically over 50 hours, which is far greater than the doubling times of CHO cells and HEK 293 cell lines; such longer doubling times are undesirable for bio-production applications in industry. To date it is not known how postnatal stem cells could be transformed into immortalized cell lines that could achieve doubling times comparable to cell lines such as CHO cells and HEK 293 cells that are currently used in industrial bio-production but have been around for more than half a century.

There is also a need for cellular immortalization approaches that are safer, better targeted and that preserve the native post-translational modification state of the bioproduct of interest as well as the plasma membrane integrity of the immortalized cell. It has been long known that infection with carcinogenic Human Papillomavirus (HPV) genotypes 16 and 18 cause cervical cancer in females and rectal cancer in both genders. However, to date exploitation of HPV genes without introducing extraneous viral genetic elements to immortalize cell targets for the production of biologics has not been achieved. HPV-16 and HPV-18 E6 and E7 genes, in concert, have been shown to regulate the cell cycle by targeting the tumor suppressor genes p53 and Rb leading to cellular proliferation and by also simultaneously preventing apoptosis of infected cells (See Tomaić et al., Cancers (Basel) 8 (10): 95, 2016). Transduction with HPV-16 E6 and E7 genes has also been shown to improve antibody production in a Sp2/0 derived cell line (See U.S. Pat. No. 8,889,410).

However, the feasibility of immortalizing somatic and/or stem cells via the insertion of E6 and E7 sequences using precision gene-editing approaches such as CRISPR has not been shown, much less in a fashion that can reduce the doubling time of the resulting immortalized cell to levels necessary for bio-production applications.

### SUMMARY OF THE INVENTION

Described herein is an immortalized mammalian cell configured for production of a bioproduct of interest, the cell comprising a safe harbor locus selected from an Adeno Associated Virus 1 (AAVS1) locus, a CCR5 locus, and a Rosa26 locus into which a Human Papillomavirus (HPV) E6 gene sequence and an HPV E7 gene sequence have been inserted.

In an embodiment, the immortalized mammalian cell lacks a functional p53 gene.

In an embodiment, the immortalized mammalian cell comprises polynucleotide sequence encoding a p53 protein, which polynucleotide sequence comprises one or more deletions, substitutions, and/or additions.

In an embodiment, the immortalized mammalian cell is a human cell.

In an embodiment, the immortalized mammalian cell is an immortalized stem cell or an immortalized somatic cell. For example, the immortalized mammalian cell may be an immortalized mesenchymal stem cell, an immortalized cord blood stem cell, or an immortalized postnatal somatic stem cell.

In an embodiment, the immortalized mammalian cell is free of extraneous viral genetic elements.

In an embodiment, the immortalized mammalian cell further comprises a heterologous polynucleotide encoding a bioproduct of interest.

In an embodiment, the heterologous polynucleotide encoding the bioproduct of interest may be inserted into a CCR5 gene locus. Also described herein is such an immortalized mammalian cell, wherein the CCR5 gene locus comprises a safe harbor region and the heterologous polynucleotide encoding the bioproduct of interest has been inserted into the safe harbor region.

In an embodiment, the immortalized mammalian cell comprises an AAVS1 locus, wherein the AAVS1 locus is an endogenous AAVS1 locus, optionally intron 1 of the protein phosphatase 1, regulatory subunit 12C (PPP1R12C) gene on human chromosome 19.

In an embodiment, the immortalized mammalian cell comprises an AAVS1 locus, wherein the AAVS1 locus is an exogenous AAVS1 safe harbor locus.

In an embodiment, the immortalized mammalian cell has a doubling time of 20-25 hours.

In an embodiment, the immortalized mammalian cell has a normal (wild-type) karyotype.

In an embodiment, the immortalized mammalian cell comprises an HPV-16 E6 gene sequence and an HPV-16 E7 gene sequence. For example, in an embodiment, the immortalized mammalian cell as described herein may comprise an internal ribosome entry site (IRES) between the HPV-16 E6 gene sequence and the HPV-16 E7 gene sequence.

Also described herein is a stable cell line configured for expression of a transgene of interest, which cell line comprises an AAVS1 locus containing an HPV E6 gene sequence and an HPV E7 gene sequence, and optionally a modified p53 sequence.

Also described herein is a method for immortalizing a mammalian cell which comprises obtaining a somatic mammalian cell comprising an AAVS1 locus, inserting an HPV E6 gene sequence and an HPV E7 gene sequence at the AAVS1 locus, inhibiting p53 activity in the mammalian cell, and allowing the cell to proliferate under conditions to produce an immortalized mammalian cell.

In an embodiment, HPV E6 and HPV E7 insertion is performed in the absence of additional HPV viral elements.

In an embodiment, inhibiting p53 activity comprises genetically modifying endogenous p53 to disrupt p53 expression and/or function. For example, in methods as contemplated herein, the endogenous p53 may be deleted.

Also disclosed herein is a method of producing a bioproduct of interest, comprising obtaining an immortalized mammalian cell as described above, transfecting the cell with a polynucleotide encoding the bioproduct of interest, and culturing the cell under conditions to produce the bioproduct of interest.

In an embodiment, the method of producing the bioproduct of interest may comprise transiently or permanently expressing the bioproduct of interest from a transgene.

In an embodiment, the bioproduct of interest is a peptide, protein, polynucleotide, or vector. Alternatively, or in addition thereto, the bioproduct of interest may be a therapeutic agent.

Also described herein is an immortalized mammalian cell configured for production of a bioproduct of interest, which cell comprises a CCR5 gene locus safe harbor into which a polynucleotide of interest has been inserted, an E6 gene sequence, an E7 gene sequence, and optionally a modified p53 sequence. In an embodiment, a part or all of the CCR5 gene in such an immortalized mammalian cell may have been replaced by the polynucleotide of interest.

In an embodiment, expression of the polynucleotide of interest in the immortalized cell is under the control of an exogenous promoter. For example, the exogenous promoter may be an EF-1α promoter.

In an embodiment, the polynucleotide of interest may encode a peptide or protein. In an embodiment, the polynucleotide of interest may encode an antibody.

In an embodiment, the polynucleotide of interest may comprise an E1 gene sequence. In such an embodiment, the immortalized cell may be configured for production of one or more viral vectors.

Also disclosed herein is a method for producing a mammalian cell for production of a bioproduct of interest, which method comprises obtaining a somatic mammalian cell comprising an AAVS1 locus, inserting an HPV E6 gene sequence and an HPV E7 gene sequence at the AAVS1 locus, allowing the cell to proliferate under conditions to produce an immortalized mammalian cell, and inserting a heterologous polynucleotide encoding a bioproduct of interest in the AAVS1 locus.

Also disclosed herein is an immortalized mammalian cell produced by a method as above. In an embodiment, such an immortalized mammalian cell may lack a functional p53 gene. In an embodiment, such an immortalized mammalian cell may comprise a polynucleotide sequence encoding a p53 protein, which polynucleotide sequence comprises one or more deletions, substitutions, and/or additions. In an embodiment, such an immortalized mammalian cell is a human cell. In an embodiment, such an immortalized mammalian cell is an immortalized stem cell or an immortalized somatic cell. For example, the immortalized mammalian cell may be an immortalized mesenchymal stem cell, an immortalized cord blood stem cell, or an immortalized postnatal somatic stem cell. In an embodiment, such an immortalized mammalian cell has a doubling time of 20-25 hours. In an embodiment, such an immortalized mammalian cell has a normal (wild-type) karyotype.

### BRIEF DESCRIPTION OF THE DRAWINGS

FIG. 1 Flow Chart for Method of Production of Immortalized Cell Line Configured for Production of a Bioproduct of Interest. The flow diagram shows the various possible steps involved in the production of an immortalized cell line for the production of a bioproduct of interest. Steps below the dashed line are optional.

FIGS. 2A-C Exemplary Constructs for Production of Immortalized Cell Line. FIG. 2A shows a CCR5 Knock In vector. FIG. 2B shows a pCas Guide Vector for use with the CCR5 Knock In Vector. FIG. 2C shows an AAVS1 Donor Vector. Relevant polynucleotide elements and restriction sites are shown in each of FIGS. 2A, 2B, and 2C.

FIG. 3 Montage of Parental Postnatal Stem Cells Before and After Immortalization. The left panel shows parent AM-MSC and CBDUSSC images respectively under phase contrast. The right panel shows the corresponding images after immortalization with the E6/E7 genes.

FIG. 4 Comparison of CHO, HEK 293, and Immortalized Cell Comprising HPV E6 and E7 Sequences. A table showing the advantages of an immortalized cells obtained by methods as disclosed herein, including doubling time, ploidy, and post-translational modification (PTM) state.

FIG. 5 Commassie Blue stain of Cellular Proteins Secreted by Immortalized Cell Comprising HPV E6 and E7 Sequences. FIG. 5 shows a Commassie blue stain of cellular proteins secreted by cultured immortalized cord blood derived unrestricted somatic stem cells (CBDUSSCs) subjected to purification of human TPO. Immortalized CBDUSSCs that were transformed by CRISPR-based gene editing using E6/E7 gene transcripts were transiently transfected with a plasmid expressing a gene that encodes for TPO. Lane 1: molecular weight markers. Lane 2: unpurified supernatant of tissue culture media. Lane 3: purified TPO after ion-exchange separation.

FIG. 6 Doubling Times. Table of doubling times shows that doubling times are significantly and unexpectedly reduced in CBUSSCs and AMMSCs when such cells (1) are deficient in p53, (2) express HPV E6 and HPV E7 sequences from a safe harbor locus, or (3) both.

FIGS. 7A-7D Embodiment of Immortalized Cells Expressing ACE2-R and TMPRSS2. In an embodiment, these immortalized cells are shown to naturally express ACE2-R and TMPRSS2, which are necessary receptors/proteins required for producing coronavirus vaccines. FIG. 7A shows cord blood derived unrestricted somatic stem cells expressing ACE2-R. FIG. 7B shows amniotic mesenchymal stem cells expressing ACE2-R. FIG. 7C shows amniotic mesenchymal stem cells expressing TMPRSS2. FIG. 7D shows cord blood derived unrestricted somatic stem cells expressing TMPRSS2.

FIG. 8 Gene map of B.1.1.7 genome. FIG. 8 shows a gene map of the Covid B.1.1.7 genome cloned into the pVC-604 plasmid.

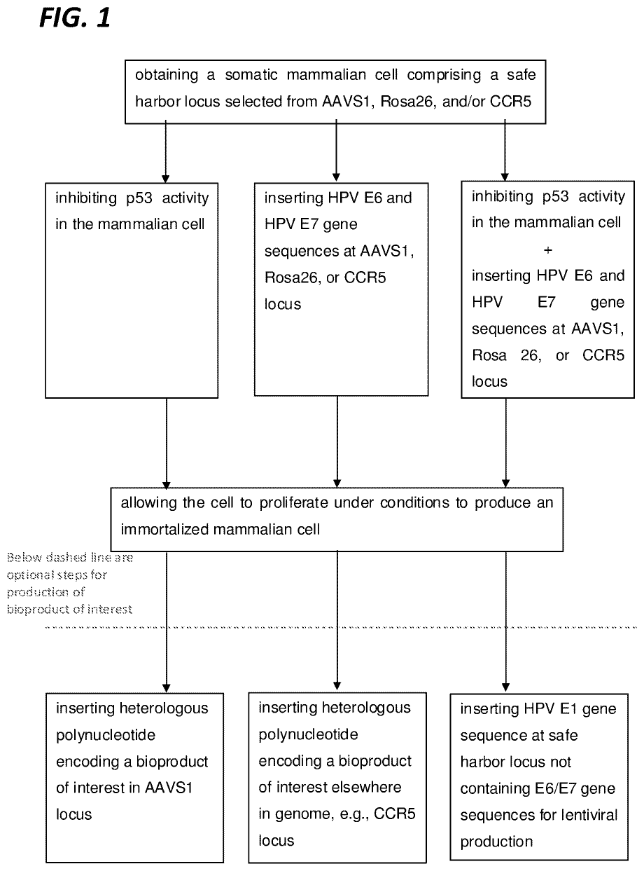

### DETAILED DESCRIPTION OF THE INVENTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by one of ordinary skill in the art to which this invention belongs. The terminology used in the description herein is for describing particular embodiments only and is not intended to be limiting. As used in the description and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless indicated to the contrary, the numerical parameters set forth in the following specification and attached claims are approximations that may vary depending upon the desired properties sought to be obtained and thus may be modified by the term “about”. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Applicant also contemplates ranges derived from data points and express ranges disclosed herein.

### Advantages and Features

Other features and advantages will be evident to those of skill in the art. For example:
Reduced doubling time (60% of parent)
Native post-translation modifications
Euploid
Permanent transfer of immortalization genes
Free of additional genetic modifications, including viral insertions and/or viral surface proteins/protein derivatives
Insertion in the genome with IRES allows for 1:1 ratio of E6/E7 expression.
Parental cell line source is available for further production of immortalized cells

### Initial Isolation of Cells, Initial Culturing Are Not Particularly Limited.

The types of cells contemplated as a starting material for obtaining an immortalized cell are not particularly limited and include somatic, stem, primary, mammalian (including mouse, hamster, rat, human, monkey). Preferably the cell is any amniotic and/or postnatal stem cell, including for example, amniotic mesenchymal stem cells and multipotent stem cells. Preferably the cell-before and after immortalization—allows for the same post-translational modifications as the wild-type cell from which it derived. Optionally, the cell is capable of post-translational modifications to the transgene of interest which are the same as the host cell from which the transgene is derived. Other types of cells contemplated as a starting material include somatic stem cells such as bone marrow and adipose-derived mesenchymal stem cells; and somatic cells such as foreskin fibroblasts, human keratinocytes and hepatocytes, Type II alveolar epithelial cells, cardiomyocytes, and renal tubular cells.

### Generation of DNA for Transfection of Isolated Cell

To obtain a somatic or stem cell configured for production of bioproducts, the cell may contain one or more of the following possible loci/safe harbors for integration of the E6 and E7 sequences: AAVS1, Rosa26, or CCR5. For example, the E6/E7 sequences may be integrated into AAVS1 safe harbor locus, as described in the Examples below. Alternatively, the E6/E7 sequence may be integrated at the Rosa26 or CCR5 loci. The E6 and E7 sequences need not be inserted at the same loci. The loci for insertion may be native to the cell or exogenous loci integrated into the genome. The E6/E7 sequences may be inserted into the respective gene or locus or may replace all or a portion of the locus.

In an embodiment, the AAVS1 locus may be an endogenous AAVS1 locus, optionally intron 1 of the protein phosphatase 1, regulatory subunit 12C (PPP1R12C) gene on human chromosome 19.

In an embodiment, the CCR5 locus may be an endogenous CCR5 locus. In an embodiment, the CCR5 gene locus may comprise a safe harbor region and the heterologous polynucleotide encoding the bioproduct of interest may be inserted into the safe harbor region.

In an embodiment, the Rosa26 locus may be an endogenous Rosa26 locus. In an embodiment, the Rosa26 gene locus may comprise a safe harbor region and the heterologous polynucleotide encoding the bioproduct of interest may be inserted into the safe harbor region.

In an embodiment, an internal ribosome entry site (IRES) may be placed between the HPV-16 E6 gene sequence and the HPV-16 E7 gene sequence.

Preferably the E6 and E7 genes are expressed in a 1:1 ratio, although other ratios are possible. Generally speaking, unless empirically determined, 1:1 ratios are preferred. This is because 1 molecule of the E6 protein and 1 molecule of the E7 protein may form a cooperative complex, which then may interact together to immortalize the cells. In cells in which E6 and E7 are expressed in a ratio other than a 1:1 ratio, one of the cognate partners may be lacking and immortalization may be suboptimal.

The E6/E7 sequences may be derived from human papillomavirus type 16 (HPV-16) or HPV-18; the E6/E7 sequences need not be limited to these HPV types.

In an embodiment, an exemplary E6 nucleotide sequence may be a nucleotide sequence which has at least 75%, 80%, 85%, 90%, 91%, 92%, 93%, 94%, 95%, 96%, 97%, 98%, or 99% identity to the to the HPV-16 E6 coding sequence (SEQ ID NO: 1). In an embodiment, an exemplary E6 amino acid sequence may be an amino acid sequence which has at least 75%, 80%, 85%, 90%, 91%, 92%, 93%, 94%, 95%, 96%, 97%, 98%, or 99% identity to the HPV-16 E6 amino acid sequence (SEQ ID NO: 2).

In an embodiment, an exemplary E7 nucleotide sequence may be a nucleotide sequence which has at least 75%, 80%, 85%, 90%, 91%, 92%, 93%, 94%, 95%, 96%, 97%, 98%, or 99% identity to the to the HPV-16 E7 coding sequence (SEQ ID NO: 3). In an embodiment, an exemplary E7 amino acid sequence may be an amino acid sequence which has at least 75%, 80%, 85%, 90%, 91%, 92%, 93%, 94%, 95%, 96%, 97%, 98%, or 99% identity to the HPV-16 E7 amino acid sequence (SEQ ID NO: 4).

Preferably the E6/E7 sequences are expressed from a constitutive or ubiquitous or mammalian promoter.

Promoters for use in the starting material and/or in the immortalized cells contemplated herein are not particularly limited, and may be selected from one or more of: elongation factor-1 alpha (EF-1α) promoter, cytomegalovirus (CMV) promoter, CAG promoter (a combination of the CMV immediate early enhance and the chicken β-actin promoter). One of skill in the art may select a suitable promoter from those known in the art depending on the type of cell being immortalized, the bioproduct to be produced, and the like. For example, the promoter may be an inducible promoter or a tissue-specific promoter.

### Suppression of p53

Optionally, the immortalized cell will lack functional p53.

The suppression of p53 activity is not particularly limited in the means by which it is achieved. For example, activity of p53 may be suppressed by the following manipulations to the cell: knock out of the p53 gene, generation of one or more mutations in the p53 sequence, administration of pharmacological inhibitors, reduction of p53 protein expression, or by introduction of an interfering protein that competes with or interacts with p53 to suppress its activity (e.g., mdm2, including modified mdm2 or mdm2-mimetics that do not dissociate from endogenous p53 protein in response to cellular injury).

Reduction of p53 protein expression may be achieved by reducing the expression of p53 mRNA or by suppression of p53 gene transcription. Genetic modifications such as gene deletion, insertion of stop codons, or introduction of one or more nucleotide insertions, deletions, substitutions, or additions into the p53 gene in the genome can result in a p53 protein having reduced activity, when the modified gene is transcribed and translated. Genetic modification to the native promoter for p53 to reduce p53 expression is also contemplated.

Pharmacologic inhibition of p53 activity is also contemplated. Pharmacological inhibitors of p53 include, but are not limited to, pifithrin-α, cyclic pifithrin-α, pifithrin-μ, RITA, SJ 172550, and/or nutlin-3, and pharmaceutically acceptable salts thereof, such as pifithrin-α hydrobromide.

Knock-down approaches include, but are not limited to, administration of antisense oligonucleotides, siRNA, RNAi, transfection of genes encoding for antisense oligonucleotides, siRNA, RNAi, all of which can be administered with or without the assistance of virion vectors, nucleofection, electroporation, or lipofectamine transfection reagents to promote cellular uptake of the nucleic acids.

### Culturing Post Transfection and Doubling Times

Doubling time could be as low as 18 hours or as high as 32 hours. Doubling time decreases as cells are seasoned out over a period of 4 months. For example, cells comprising E6/E7 sequences, cells in which p53 activity has been suppressed, or cells configured to express E6 and E7 as well as to suppress p53, may be cultured by methods known to those of skill in the art, including, for example, in the presence of an antibiotic to select for cells which have been successfully transfected. The culture conditions may such that allow for unconstrained proliferation and/or growth over the course of two, three, four, or five weeks, or two, three, or four months, or until such time that the cells achieve a decreased doubling time compared to the native cell from which they were derived.

In a preferred embodiment, doubling times of the immortalized host cells may be 20, 21, 22, 23, 24, or 25 hours, or in the range of time from 21-24 hours with an endpoint as low as 18 hours and as high as 30 or 32 hours. Alternatively, reduced doubling time may be determined relative to the cell prior to configuration for production of the bioproduct. For example, if the starting cell is an amniotic membrane mesenchymal stem cell having a doubling time of 52-60 hours, the immortalized cells obtained by methods as described herein may achieve doubling times reduced by 30, 35, 40, 45, 50, 55, or 60%, compared to the corresponding native cell from which it is derived.

Bioproducts Capable of Production with Immortalized Host Cells of the Invention

Bioproducts for production with immortalized cells of the invention are not particularly limited and include peptides, protein, antibodies, and vectors including viral vectors. Bioproducts may also include nucleic acids, and RNP complexes, and viral like particles (VLPs).

Antibodies contemplated for production include whole antibodies and functionally active fragments or derivatives thereof and may be, but are not limited to, monoclonal, multi-valent, multi-specific, bispecific, fully human, humanized or chimeric antibodies, domain antibodies e.g. VH, VL, VHH, single chain antibodies, Fab fragments, Fab′ and F(ab′)₂ fragments and epitope-binding fragments of any of the above. Antibody fragments and methods of producing them are well known in the art. Antibodies contemplated for production herein also include immunoglobulin molecules and immunologically active portions of immunoglobulin molecules, i.e., molecules that contain an antigen binding site that specifically binds an antigen. The immunoglobulin molecules of the invention can be of any class (e.g. IgG, IgE, IgM, IgD, and IgA) or subclass of immunoglobulin molecule. It will also be understood by one of skill in the art that antibodies may undergo a variety of posttranslational modifications. The type and extent of these modifications may depend on the host cell line used to express the antibody as well as the culture conditions. Such modifications may include variations in glycosylation, methionine oxidation, diketopiperazine formation, aspartate isomerization, asparagine deamidation, and loss of a carboxy-terminal basic residue (such as lysine or arginine).

A ribonucleoprotein (RNP) is a complex of ribonucleic acid and RNA-binding protein. Host cells as contemplated herein may produce a single component of the RNP complex or the entire complex.

Viral-like particles (VLPs) are multiprotein structures that mimic the organization and conformation of authentic native viruses but lack a viral genome. Thus, VLPs are potentially safer and cheaper vaccine candidates compared to their corresponding native viruses.

Methods for Production of Bioproducts with Immortalized Cells of the Invention

Transfecting one or more of the immortalized cells to generate a cell line expressing a protein of interest may include transfection with a gene encoding the protein or other bioproduct under control of a ubiquitous or mammalian promoter that causes expression in the host cell line. The gene of interest may be transiently transfected by way of, for example, a protein expression cassette. Alternatively or subsequently, permanent or stable transfection may be performed that integrates the gene of interest or a marker gene into the genome of the cell line. Transfection may be performed with a liposome-based reagent, calcium phosphate, electroporation, or infection with an adenovirus-, retrovirus- or lentivirus-based vector.

Once the cell has been configured for production of the bioproduct of interest, the cell may be cultured under conditions yielding small-scale or large-scale production.

Methods for production of viruses and vaccines are not particularly limited. For example, such techniques may include techniques as disclosed by Benskey et al. (Methods Mol Biol. 1382:107-14, 2016 doi: 10.1007/978-1-4939-3271-9_8), which is incorporated by reference herein in its entirety.

Methods for production of antibodies are also not particularly limited and include techniques as disclosed by Trill et al. (Curr Opin Biotechnol, 6(5): 553-60, 1995 doi: 10.1016/0958-1669 (95) 80092-1), which is incorporated by reference herein in its entirety.

Methods for production of peptides and proteins for therapeutic use are also not particularly limited and include techniques as disclosed by Kim et al. (Appl Microbiol Biotechnol. 93(3): 917-30, 2012 doi: 10.1007/s00253-011-3758-5), which is incorporated by reference herein in its entirety.

Methods for production of RNA and/or RNPs and/or VLPs are also not particularly limited and include techniques as disclosed by Zeltins (Mol Biotechnol. 53(1): 92-107, 2013. doi: 10.1007/s12033-012-9598-4), which is incorporated by reference herein in its entirety.

### EXAMPLES

### Example 1

### Generation of Starting Cells and Vector Constructs-AAVS1

HPV-16 E6 and E7 sequences were obtained from PubMed and each was synthetically produced and cloned into a pUC-19 cloning vector. The sequences were then subjected to a polymerase chain reaction (PCR) and an internal ribosome entry site (IRES) was introduced between the E6 and E7 open reading frames. This allows for simultaneous expression of both genes while maintaining an equivalent protein molar ratio within a cell. Then the modified construct, which now consisted of the E6 gene—IRES—E7 gene was subcloned into each of two mammalian expression vectors configured to target the Adeno Associated Virus 1 (AAVS1) Safe Harbor Locus. Once this expression cassette was cloned into both vectors, they were subjected to DNA sequencing and purified DNA containing these genes was made for introduction into cells. Both vectors were then quantitated using absorbance spectroscopy at 280 nm and transfected into target cells using a lipid-based reagent. At the end of 72 hours, both genes, which are flanked by homologous recombination arms were integrated into the AAVS1 locus along with the gene coding for the antibiotic resistance gene, Blasticidin. Cells were then screened for blasticidin expression to achieve a pure population.

### Generation of Immortalized Cells—AAVS1

Isolated and actively growing AM-MSC and CBDUSSC cells were transfected with either the mammalian expression vector containing these genes or a CRISPR vector containing these genes to obtain permanent or stable cell lines. DNA was introduced into cells containing a lipid-based approach that allowed for high non-viral transduction and low cell mortality. After 72 hours, each cell line was challenged with antibiotics that were toxic to each cell type unless the cell had successfully acquired the expression cassette coded for in each vector type.

In addition, the p53 gene was knocked out by introducing a frame shift mutation in the coding region of the Open Reading Frame using CRISPR. Briefly, an “Indel” mutation was introduced which deleted 3 nucleotides that coded for the native p53 protein and these were replaced by stuffer sequences that led to premature termination of protein translation.

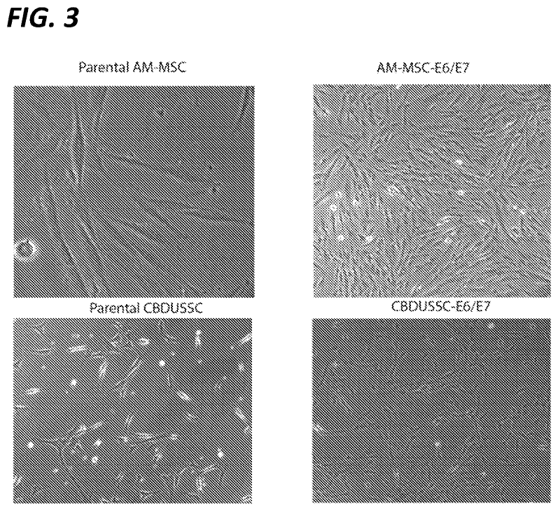

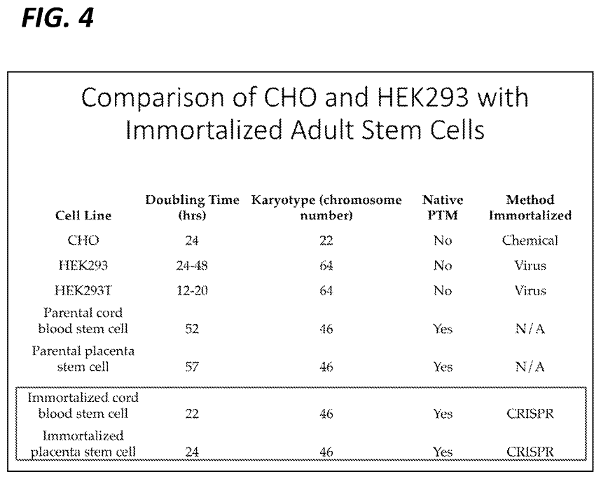

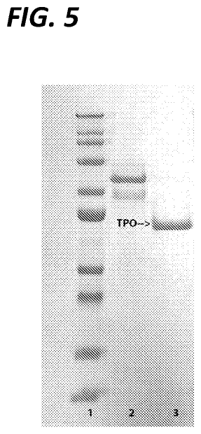

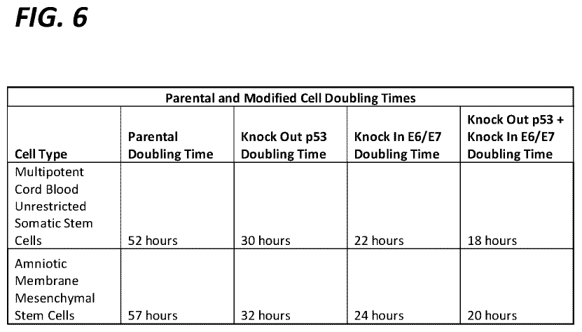

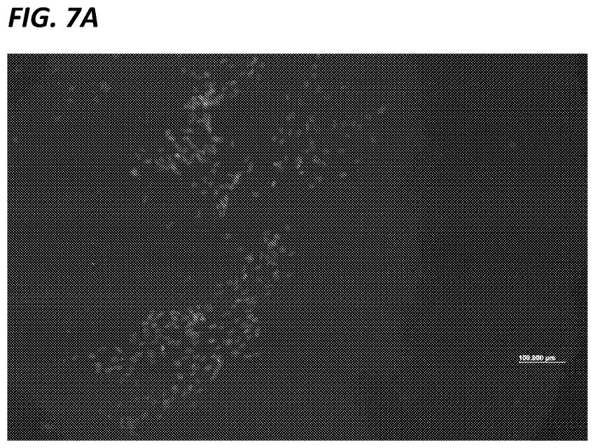

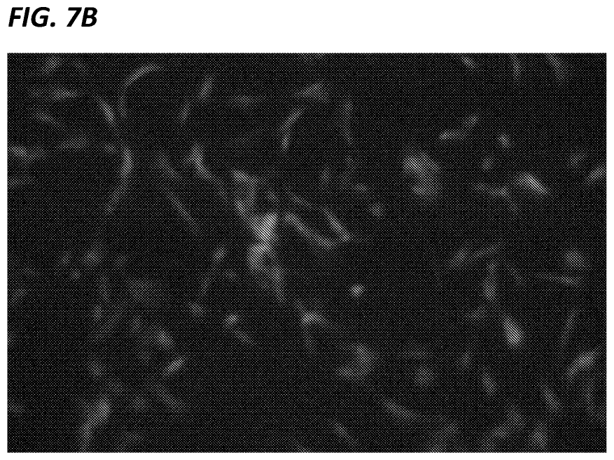

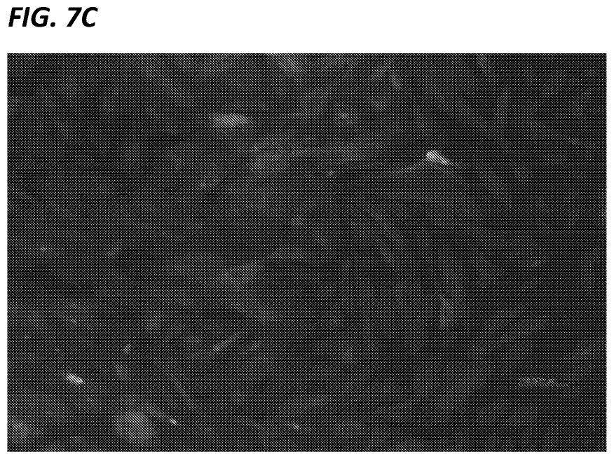

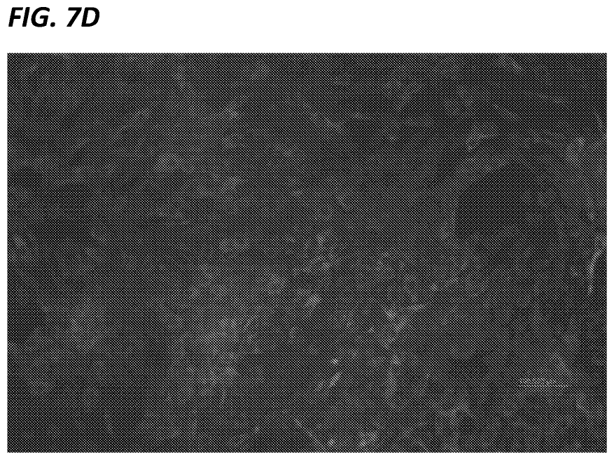

After about a week, distinct colonies were visible by microscopy that showed cells with altered shape/morphology (FIG. 3).

### Example 2

### Generation of Immortalized Cells—CCR5

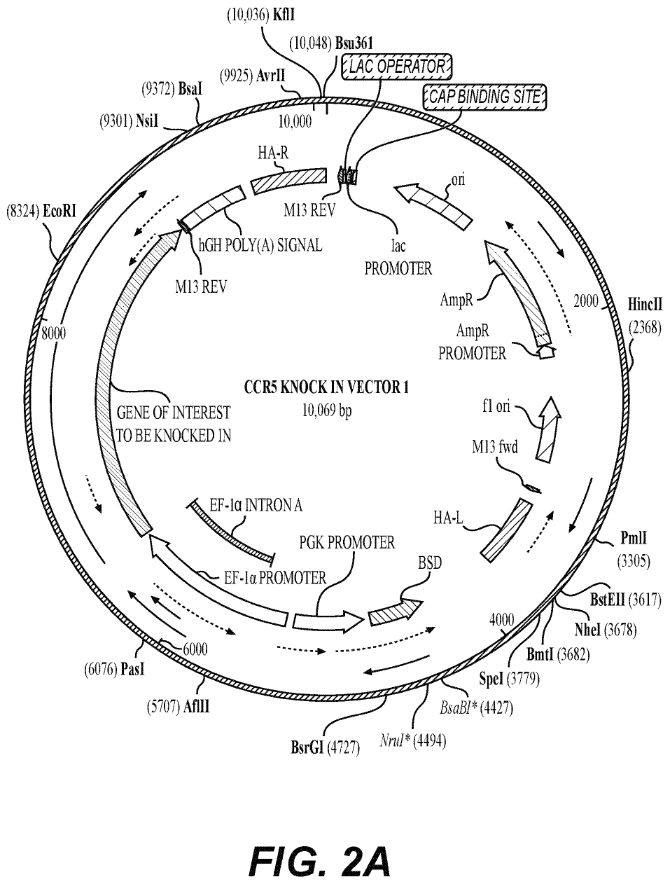

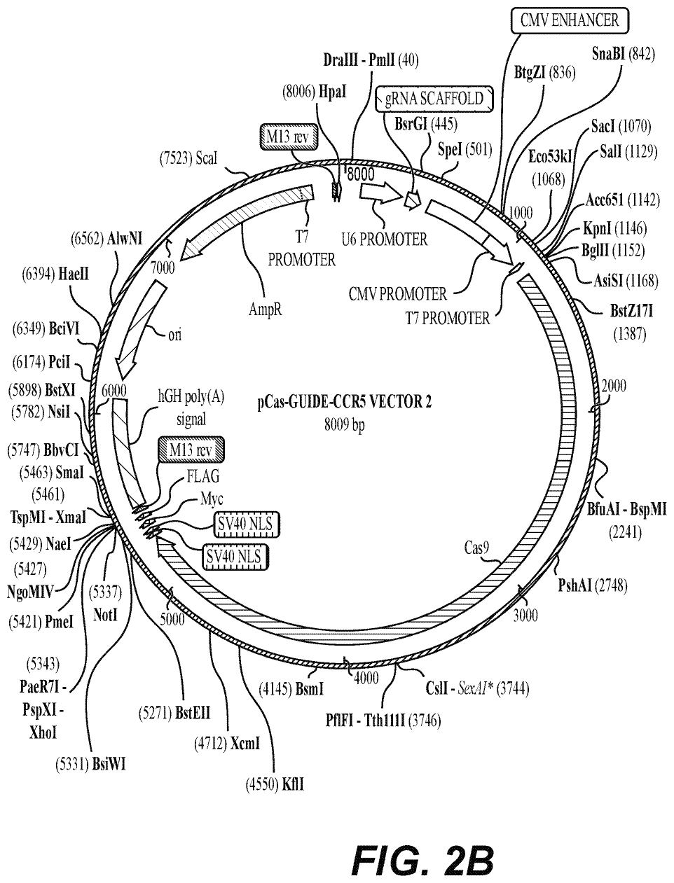

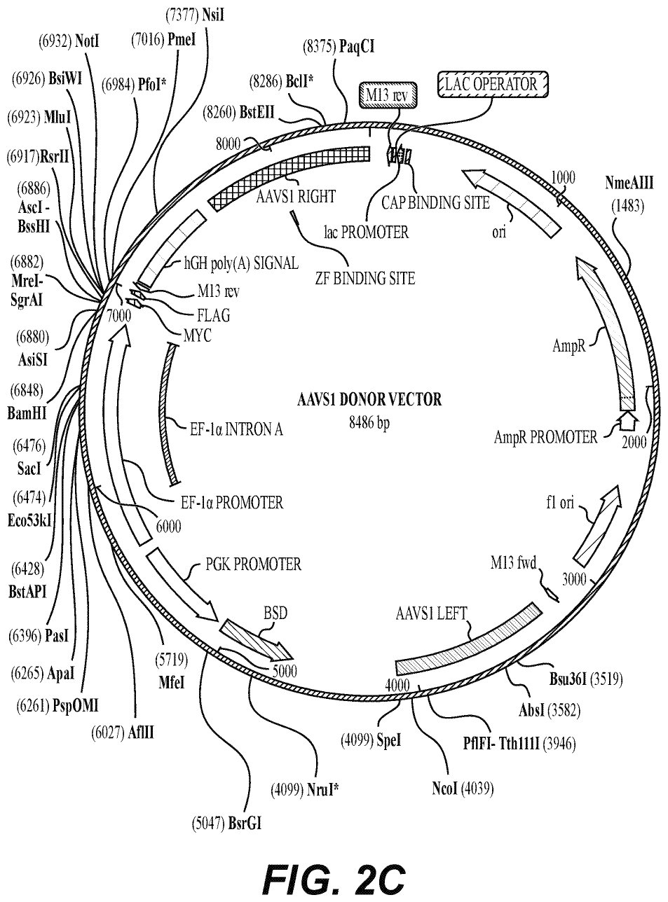

We used a two-vector knock in methodology where one vector contained the left and right homologous recombination arms flanking the gene of interest. The second vector, the guide vector, contained the coding sequence for the cas9 nuclease enzyme along with the guide PAM sequence to direct the gene to the appropriate safe harbor location. The vector diagrams are set forth in FIGS. 2A-2B.

In addition, the p53 gene was knocked out.

The sequence information is as follows:

| | |
|---|---|
| PAM Sequence | 5′GGAGAGCTTGGCTCTGTTGGGGG3′ |
| | (SEQ ID NO: 5) |
| Left Homologous | 5′CCGGCCATTTCACTCTGACTACATC |
| Recombination Arm | ATGTCACCAAACATCTGATGGTCTTGC |
| | CTTTTAATTCTCTTTTCGAGGACTGAG |
| | AGGGAGGGTAGCATGGTAGTTAAGAGT |
| | GCAGGCTTCCCGCATTCAAAATCGGTT |
| | GCTTACTAGCTGTGTGGCTTTGAGCAA |
| | GTTACTCACCCTCTCTGTGCTTCAAGG |
| | TCCTTGTCTGCAAAATGTGAAAAATAT |
| | TTCCTGCCTCATAAGGTTGCCCTAAGG |
| | ATTAAATGAATGAATGGGTATGATGCT |
| | TAGAACAGTGATTGGCATCCAGTATGT |
| | GCCCTCGAGGCCTCTTAATTATTACTG |
| | GCTTGCTCATAGTGCATGTTCTTTGTG |
| | GGCTAACTCTAGCGTCAATAAAAATGT |
| | TAAGACTGAGTTGCAGCCGGGCATGGT |
| | GGCTCATGCCTGTAATCCCAGCATTCT |
| | AGGAGGCTGAGGCAGGAGGATCGCTTG |
| | AGCCCAGGAGTTCGAGACCAGCCTGGG |
| | CAACATAGTGTGATCTTGTATCTATAA |
| | AAATAAACAAAATTAGCTTGGTGTGGT |
| | GGCGCCTGTAGTCCCCAGCCACTTGGA |
| | GGGGTGAGGTGAGAGGATTGCTTGAGC |
| | CCGGGATGGTCCAGGCTGCAGTGAGCC |
| | ATGATCGTGCCACTGCACTCCAGCCTG |
| | GGCGACAGAGTGAGACCCTGTCTCACA |
| | ACAACAACAACAACAACAAAAAGGCTG |
| | AGCTGCACCATGCTTGACCCAGTTTCT |
| | TAAAATTGTTGTCAAAGCTTCATTCAC |
| | TCCATGGTGCTATAGAGCACAAGATTT |
| | TATTTGGTGAGATGGTGCTTTCATGAA |
| | TTCCCCCA3′ |
| | (SEQ ID NO: 6) |
| Right Homologous | 5′ACAGAGCCAAGCTCTCCATCTAGT |
| Recombination Arm | GGACAGGGAAGCTAGCAGCAAACCTT |
| | CCCTTCACTACAAAACTTCATTGCTTG |
| | GCCAAAAAGAGAGTTAATTCAATGTAG |
| | ACATCTATGTAGGCAATTAAAAACCTA |
| | TTGATGTATAAAACAGTTTGCATTCAT |
| | GGAGGGCAACTAAATACATTCTAGGAC |
| | TTTATAAAAGATCACTTTTTATTTATG |
| | CACAGGGTGGAACAAGATGGATTATCA |
| | AGTGTCAAGTCCAATCTATGACATCAA |
| | TTATTATACATCGGAGCCCTGCCAAAA |
| | AATCAATGTGAAGCAAATCGCAGCCCG |
| | CCTCCTGCCTCCGCTCTACTCACTGGT |
| | GTTCATCTTTGGTTTTGTGGGCAACAT |
| | GCTGGTCATCCTCATCCTGATAAACTG |
| | CAAAAGGCTGAAGAGCATGACTGACAT |
| | CTACCTGCTCAACCTGGCCATCTCTGA |
| | CCTGTTTTTCCTTCTTACTGTCCCCTT |
| | CTGGGCTCACTATGCTGCCGCCCAGTG |
| | GGACTTTGGAAATACAATGTGTCAACT |
| | CTTGACAGGGCTCTATTTTATAGGCTT |
| | CTTCTCTGGAATCTTCTTCATCATCCT |
| | CCTGACAATCGATAGGTACCTGGCTGT |
| | CGTCCATGCTGTGTTTGCTTTAAAAGC |
| | CAGGACGGTCACCTTTGGGGTGGTGAC |
| | AAGTGTGATCACTTGGGTGGTGGCTGT |
| | GTTTGCGTCTCTCCCAGGAATCATCTT |
| | TACCAGATCTCAAAAAGAAGGTCTTCA |
| | TTACACCTGCAGCTCTCATTTTCCATA |
| | CAGTCAGTATCAATTCTGGAAGAATTT |
| | CCAGACATTAAAGATAGTCATCTTGGG |
| | GCTGGTCCTGCCGCTGCTTGTCATGGT |
| | CATCTGCTACTCGGGAA3′ |
| | (SEQ ID NO: 7) |

### Results

The gene of interest was cloned using seamless infusion cloning into Vector 1. 100,000 human stem cells were co-transfected with equimolar quantities of vector 1 and vector 2 (0.5 micrograms each). Gene expression and positive colonies were selected by screening the cells with blasticidin antibiotic over a 3-week period. Cells were considered positively selected when discreet colonies appeared and continued to divide under antibiotic selection.

### Example 3

### Production of Template for Creation of Live Attenuated Strains in Immortalized Human Somatic Stem Cells

The full-length genome of Covid UC strain (B.1.1.7) was generated and cloned into a PVC-604 vector and maintained in Saccharomyces cerevisiae (strain VL6-48N). pVC-604 functions as a Yeast Artificial Chromosome (YAC) owing to its stability in maintaining large genomic DNA sequences (>25 kb). The pVC-604 vector is suitable for TAR cloning since it contains the yeast origin of replication. Using synthetic biology and in silico software, 5 overlapping DNA fragments were prepared and ligated in frame. Two key genetic modifications were made and included: 1) insertion of a T-7 RNA Polymerase Promoter upstream at the 5′ end along with a Pac I endonuclease cleavage site and 2) a Mlu I restriction endonuclease cleavage site downstream of the 3′ poly A sequence along with overlapping sequences necessary for TAR cloning with the pVC604 plasmid.

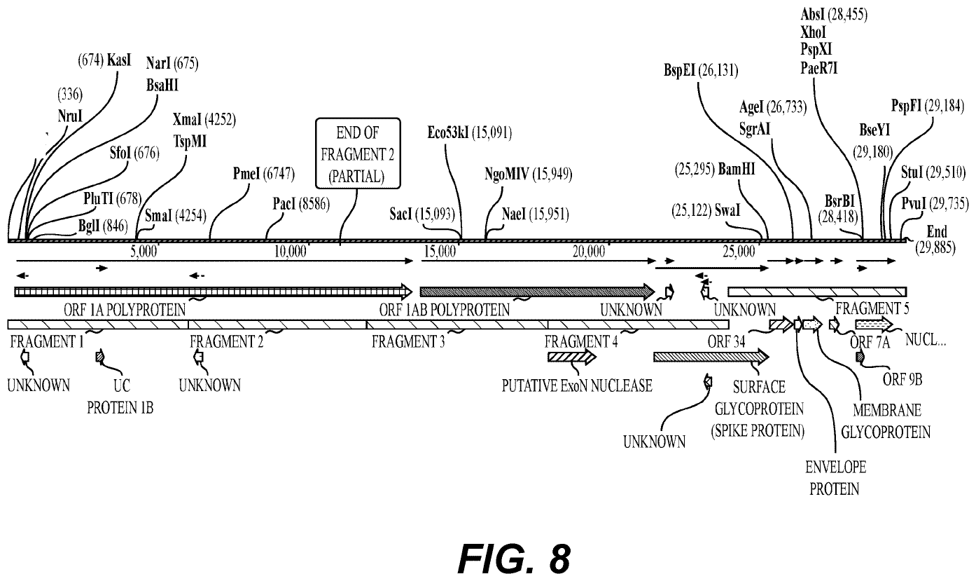

A gene map of the B.1.1.7 genome cloned into the pVC-604 plasmid is shown in FIG. 8.

This template can be genetically modified for creating live attenuated strains by deletions of virulence genes using PCR; DNA amplification was done by omitting the fragment to be deleted and creating overlapping fragments that span the deleted genomic DNA sequence or by site-directed mutagenesis. This vector also has a bacterial origin of replication that allows for bacterial expansion and maintenance. Moreover, this template can be used to create known variants by site-directed mutagenesis.

Sequence Listing (Free Text)
HPV 16/18 E6 nucleotide sequence
(SEQ ID NO: 1)
ATGCACCAAAAGAGAACTGCAATGTTTCAGGACCCACAGGAGCGA
CCCAGAAAGTTACCACAGTTATGCACAGAGCTGCAAACAACTATA
CATGATATAATATTAGAATGTGTGTACTGCAAGCAACAGTTACTG
CGACGTGAGGTATATGACTTTGCTTTTCGGGATTTATGCATAGTA
TATAGAGATGGGAATCCATATGCTGTATGTGATAAATGTTTAAAG
TTTTATTCTAAAATTAGTGAGTATAGACATTATTGTTATAGTTTG
TATGGAACAACATTAGAACAGCAATACAACAAACCGTTGTGTGAT
TTGTTAATTAGGTGTATTAACTGTCAAAAGCCACTGTGTCCTGAA
GAAAAGCAAAGACATCTGGACAAAAAGCAAAGATTCCATAATATA
AGGGGTCGGTGGACCGGTCGATGTATGTCTTGTTGCAGATCATCA
AGAACACGTAGAGAAACCCAGCTGTAA
HPV 16/18 E6 amino acid sequence
(SEQ ID NO: 2)
MHQKRTAMFQDPQERPRKLPQLCTELQTTIHDIILECVYCKQQLL
RREVYDFAFRDLCIVYRDGNPYAVCDKCLKFYSKISEYRHYCYSL
YGTTLEQQYNKPLCDLLIRCINCQKPLCPEEKQRHLDKKQRFHNI
RGRWTGRCMSCCRSSRTRRETQL*
HPV 16/18 E7 nucleotide sequence
(SEQ ID NO: 3)
ATGCATGGAGATACACCTACATTGCATGAATATATGTTAGATTT
GCAACCAGAGACAACTGATCTCTACTGTTATGAGCAATTAAATG
ACAGCTCAGAGGAGGAGGATGAAATAGATGGTCCAGCTGGACAAG
CAGAACCGGACAGAGCCCATTACAATATTGTAACCTTTTGTTGCA
AGTGTGACTCTACGCTTCGGTTGTGCGTACAAAGCACACACGTAG
ACATTCGTACTTTGGAAGACCTGTTAATGGGCACACTAGGAATTG
TGTGCCCCATCTGTTCTCAGAAACCATAA
HPV 16/18 E7 amino acid sequence
(SEQ ID NO: 4)
MHGDTPTLHEYMLDLQPETTDLYCYEQLNDSSEEEDEIDGPAGQA
EPDRAHYNIVTFCCKCDSTLRLCVQSTHVDIRTLEDLLMGTLGIV
CPICSQKP*

What is claimed is:

1. An immortalized mammalian cell configured for production of a bioproduct of interest, the cell comprising a safe harbor locus selected from the group consisting of an Adeno Associated Virus 1 (AAVS1) locus, a CCR5 locus, and a Rosa26 locus into which a Human Papillomavirus (HPV) E6 gene sequence and an HPV E7 gene sequence have been inserted.

2. The immortalized mammalian cell of claim 1, which lacks a functional p53 gene.

3. The immortalized mammalian cell of claim 2, which comprises polynucleotide sequence encoding a p53 protein, which polynucleotide sequence comprises one or more deletions, substitutions, and/or additions.

4. The immortalized mammalian cell of claim 1, which is a human cell.

5. The immortalized mammalian cell of claim 1, which is an immortalized stem cell or an immortalized somatic cell.

6. The immortalized mammalian cell of claim 5, which is an immortalized mesenchymal stem cell, an immortalized cord blood stem cell, or an immortalized postnatal somatic stem cell.

7. The immortalized mammalian cell of claim 1, which is free of extraneous viral genetic elements.

8. The immortalized mammalian cell of claim 1, which further comprises a heterologous polynucleotide encoding a bioproduct of interest.

9. The immortalized mammalian cell of claim 8, wherein the heterologous polynucleotide encoding the bioproduct of interest has been inserted into a CCR5 gene locus.

10. The immortalized mammalian cell of claim 9, wherein the CCR5 gene locus comprises a safe harbor region and the heterologous polynucleotide encoding the bioproduct of interest has been inserted into the safe harbor region.

11. The immortalized mammalian cell of claim 1, wherein the AAVS1 locus is an endogenous AAVS1 locus, optionally intron 1 of the protein phosphatase 1, regulatory subunit 12C (PPP1R12C) gene on human chromosome 19.

12. The immortalized mammalian cell of claim 1, wherein the AAVS1 locus is an exogenous AAVS1 safe harbor locus.

13. The immortalized mammalian cell of claim 1, which has a doubling time of 20-25 hours.

14. The immortalized mammalian cell of claim 1, which has a normal (wild-type) karyotype.

15. The immortalized mammalian cell of claim 1, which comprises an HPV-16 E6 gene sequence and an HPV-16 E7 gene sequence.

16. The immortalized mammalian cell of claim 15, which comprises an internal ribosome entry site (IRES) between the HPV-16 E6 gene sequence and the HPV-16 E7 gene sequence.

17. A stable cell line configured for expression of a transgene of interest, which cell line comprises: an AAVS1 locus containing an HPV E6 gene sequence and an HPV E7 gene sequence, and optionally a modified p53 sequence.

18. A method for immortalizing a mammalian cell comprising: obtaining a somatic mammalian cell comprising an AAVS1 locus; inserting an HPV E6 gene sequence and an HPV E7 gene sequence at the AAVS1 locus; inhibiting p53 activity in the mammalian cell; and allowing the cell to proliferate under conditions to produce an immortalized mammalian cell.

19. The method of claim 18, wherein HPV E6 and HPV E7 insertion is performed in the absence of additional HPV viral elements.

20. The method of claim 18, wherein inhibiting p53 activity comprises genetically modifying endogenous p53 to disrupt p53 expression and/or function.

21. The method of claim 20, wherein the endogenous p53 has been deleted.

22. A method of producing a bioproduct of interest, comprising: obtaining the immortalized mammalian cell of claim 1, transfecting the cell with a polynucleotide encoding the bioproduct of interest, and culturing the cell under conditions to produce the bioproduct of interest.

23. The method of claim 22, which comprises transiently or permanently expressing the bioproduct of interest from a transgene.

24. The method of claim 22, wherein the bioproduct of interest is a peptide, protein, polynucleotide, or vector.

25. The method of claim 22, wherein the bioproduct of interest is a therapeutic agent.

26. An immortalized mammalian cell configured for production of a bioproduct of interest, the cell comprising: a CCR5 gene locus safe harbor into which a polynucleotide of interest has been inserted, an E6 gene sequence, an E7 gene sequence, and optionally a modified p53 sequence.

27. The immortalized mammalian cell of claim 26, in which a part or all of the CCR5 gene has been replaced by the polynucleotide of interest.

28. The immortalized cell of claim 26, wherein expression of the polynucleotide of interest is under the control of an exogenous promoter.

29. The immortalized cell of claim 28, wherein the exogenous promoter is an EF-la promoter.

30. The immortalized mammalian cell of claim 26, wherein the polynucleotide of interest encodes a peptide or protein.

31. The immortalized cell of claim 30, wherein the polynucleotide of interest encodes an antibody.

32. The immortalized cell of claim 26, wherein the polynucleotide of interest comprises an E1 gene sequence.

33. The immortalized cell of claim 32, wherein the cell is configured for production of one or more viral vectors.

34. A method for producing a mammalian cell for production of a bioproduct of interest comprising: obtaining a somatic mammalian cell comprising an AAVS1 locus; inserting an HPV E6 gene sequence and an HPV E7 gene sequence at the AAVS1 locus; allowing the cell to proliferate under conditions to produce an immortalized mammalian cell; and inserting a heterologous polynucleotide encoding a bioproduct of interest in the AAVS1 locus.

35. An immortalized mammalian cell produced by the method of claim 34.

36. The immortalized mammalian cell of claim 35, which lacks a functional p53 gene.

37. The immortalized mammalian cell of claim 35, which comprises polynucleotide sequence encoding a p53 protein, which polynucleotide sequence comprises one or more deletions, substitutions, and/or additions.

38. The immortalized mammalian cell of claim 35, which is a human cell.

39. The immortalized mammalian cell of claim 35, which is an immortalized stem cell or an immortalized somatic cell.

40. The immortalized mammalian cell of claim 35, which is an immortalized mesenchymal stem cell, an immortalized cord blood stem cell, or an immortalized postnatal somatic stem cell.

41. The immortalized mammalian cell of claim 35, which has a doubling time of 20-25 hours.

42. The immortalized mammalian cell of claim 1, which has a normal (wild-type) karyotype.
